(19) United States
(12) Patent Application Publication
(10) Pub. No.: US 2023/0081657 A1
(43) Pub. Date: Mar. 16, 2023

(54) SYSTEM AND METHOD FOR DETERMINING AND PREDICTING OF A MISSTEP

(71) Applicant: OWLYTICS HEAL THCARE LTD., Bnei-Atarot (IL)

(72) Inventors: Gill ZAPHRIR, Bnei Atarot (IL); Nathaniel SHIMONI, Be'er (IL); Yaron RECHER, Ramat Yishai (IL)

(73) Assignee: OWLYTICS HEAL THCARE LTD., Bnei- Atarot (IL)

(21) Appl. No.: 17/799,674

(22) PCT Filed: Feb. 11, 2021
(86) PCT No.: PCT/IL2021/050165

§ 371 Date: Aug. 14, 2022

Related U.S. Application Data
(60) Provisional application No. 62/975,786, filed on Feb. 13, 2020.

(51) Int. Cl. A61B 5/24; A61B 5/11

(52) U.S. Cl. CPC A61B 5/24; A61B 5/1117; A61B 5/02405

(57) ABSTRACT

Systems and methods of determining a mis-step of a user with gait abnormality, including: calibrating signals corresponding to motion by the user in a controlled environment to identify the gait of the user, detecting a movement of the user to determine a change from the determined gait, detecting at least one physiological signal of the user, identifying motion carried out by the user based on data from at least one wearable sensor, and determining at least one mis-step carried out by the user using a machine learning algorithm trained to determine steps based on the identified motion.

### FIELD OF THE INVENTION

The present invention relates to motion determination and analysis. More particularly, the present invention relates to systems and methods for determination and analysis of motion of elderly users or neurology patients to detect and/or predict missteps.

### BACKGROUND OF THE INVENTION

A variety of electronic devices to count steps are available today. These devices (e.g., a smart-watch) typically use built-in motion sensors to detect movement of the user that is associated with the motion of taking a step.

The common step counter (for example as utilized in a smart-watch) is usually based on cyclic motions of the users' hand wearing the counting device. These counters are built for healthy active users. However, none of the available devices is able to properly detect movement of elderly users or neurology patients and count fewer steps (or no steps at all) than were actually taken.

While detecting walking activity and counting of steps performed by young and healthy individuals is a common feature, for instance applicable in large variety of devices (e.g., in smart-watches), the use of the same devices for the detection of walking and steps counting for elderly individuals or neurology patients, is highly inaccurate, mainly due to lower signal to noise ratio (SNR), for example when elderly users have finer and/or slower movement, and high variability of signals derived by the use of various walking aids.

### SUMMARY

There is thus provided, in accordance with some embodiments of the invention, a method of determining activity status of a user with gait abnormality, the method including: receiving, by a processor, a profile of the user, detecting, by at least one wearable sensor coupled to the processor, a movement of the user, identifying, by the processor, motion carried out by the user based on the received user profile and on data from the at least one wearable sensor, and determining, by the processor, at least one step carried out by the user using a machine learning algorithm trained to determine steps based on the identified motion.

In some embodiments, the at least one wearable sensor detects signals with information indicative of physiological status selected from the group consisting of: heart rate, heart rate variability, blood pressure, foot pressure, magnetometer, gyroscope, and accelerometer in three-axis. In some embodiments, the user profile includes information selected from the group consisting o£ medical history, medication used by the user, type of walking aid, social status, age, height, and location history.

In some embodiments, the machine learning algorithm is trained to determine steps, where the training includes classifying activity statuses based on a dataset of motion data for users with gait abnormality. In some embodiments, the machine learning algorithm is trained to determine steps, where the training includes applying a regressor to determine at least one step based on varying-length sequences of signals from the at least one wearable sensor. In some embodiments, the machine learning algorithm is trained to determine steps, where the training includes creating a point-wise segmentation network for each measured point in time to determine the activity status based on the context of the measurement by the at least one wearable sensor. In some embodiments, the machine learning algorithm is trained with tagging of the activity status of the user.

In some embodiments, a statistical baseline is determined for movements of the user, based on measurements by the at least one wearable sensor. In some embodiments, similar walking patterns are clustered, the walking patterns being sampled by other users with gait abnormality. In some embodiments, abstraction of gait abnormality patterns is performed using similarity learning.

In some embodiments, the machine learning algorithm is performed using at least one of. Gated Recurrent Units (GRUs), Convolutional Neural Networks (CNNs), Long Short-Term Memory (LSTM) neural networks, Deep Auto Encoders (DAEs), attention based neural networks, transformer based neural networks, and gradient boosted decision tree algorithms.

There is thus provided, in accordance with some embodiments of the invention, a system for determination of an activity status of a user with gait abnormality, the system including: a database, including a profile of the user, at least one wearable sensor, configured to detect a movement of the user, and a processor, coupled to the database and to the at least one wearable sensor, and configured to: identify a motion carried out by the user based on the received user profile and on data from the at least one wearable sensor, and determine at least one step carried out by the user using a machine learning algorithm trained to determine steps based on the identified motion.

In some embodiments, the user profile includes information selected from the group consisting of: medical history, medication used by the user, type of walking aid, social status, age, height, and location history. In some embodiments, the at least one wearable sensor is to detect signals with information indicative of physiological status selected from the group consisting of: heart rate, heart rate variability, blood pressure, foot pressure, magnetometer, gyroscope, and accelerometer in three-axis. In some embodiments, the at least one wearable sensor detects signals sampled at frequencies in the range 1-100 Hz.

There is thus provided, in accordance with some embodiments of the invention, a method of determining a mis-step of a user with gait abnormality, the method including calibrating, by a processor, signals corresponding to motion by the user in a controlled environment to identify the gait of the user, detecting, by at least one wearable sensor coupled to the processor, a movement of the user to determine a change from the determined gait, detecting, by the at least one wearable sensor, at least one physiological signal of the user, identifying, by the processor, motion carried out by the user based on data from the at least one wearable sensor, and determining, by the processor, at least one mis-step carried out by the user using a machine learning algorithm trained to determine steps based on the identified motion.

In some embodiments, the machine learning algorithm is trained to determine steps with a dataset of motion data for users with known attributes. In some embodiments, a fall event is predicted based on an escalation of determined mis-step events.

In some embodiments, a fall risk is predicted based on detection of multiple mis-step events. In some embodiments, a fall risk is predicted based on monitoring sleep of the user. In some embodiments, a fall risk is predicted based on correlation between detected movement changes and medication changes of the user. In some embodiments, a fall risk is predicted based on monitoring behavioral abnormalities of the user.

In some embodiments, at least one mis-step by the user is detected by the at least one wearable sensor. In some embodiments, at least one fall by the user is detected by the at least one wearable sensor.

In some embodiments, abstraction of gait abnormality patterns is performed using similarity learning. In some embodiments, the machine learning algorithm is performed using at least one of: Gated Recurrent Units (GRUs), Convolutional Neural Networks (CNNs), Long Short-Term Memory (LSTM) neural networks, Deep Auto Encoders (DAEs), attention based neural networks, transformer based neural networks, and gradient boosted decision tree algorithms.

There is thus provided, in accordance with some embodiments of the invention, a system for determination of a mis-step of a user with gait abnormality, the system including: a database, including a calibrated set of signals corresponding to gait of the user, and at least one wearable sensor, configured to: detect a movement of the user, and detect at least one physiological signal of the user, and a processor, coupled to the database and to the at least one wearable sensor, and configured to: determine a change from the calibrated gait, identify a motion carried out by the user based on the received user profile and on signal from the at least one wearable sensor, and determine at least one mis-step carried out by the user using a machine learning algorithm trained to determine steps based on the identified motion.

In some embodiments, the machine learning algorithm is trained to determine steps with a dataset of motion data for users with known attributes. In some embodiments, the known attributes include information selected from the group consisting of: medical history, medication used by the user, type of walking aid, social status, age, height, and location history.

In some embodiments, the processor is configured to predict a fall event based on an escalation of determined mis-step events. In some embodiments, the processor is configured to predict a fall risk based on detection of multiple mis-step events. In some embodiments, the processor is configured to predict a fall risk based on monitoring sleep of the user. In some embodiments, the processor is configured to predict a fall risk based on correlation between detected movement changes and medication changes of the user. In some embodiments, the processor is configured to predict a fall risk based on monitoring behavioral abnormalities of the user.

In some embodiments, the at least one wearable sensor is configured to detect at least one fall by the user. In some embodiments, the at least one wearable sensor is configured to detect at least one mis-step by the user.

In some embodiments, the at least one wearable sensor includes at least one of a smartwatch motion-sensor and an insole sensor. In some embodiments, the at least one wearable sensor is configured to detect signals with information indicative of physiological status selected from the group consisting of: heart rate, heart rate variability, galvanic skin response, electrocardiogram, SpO2, barometric pressure, magnetometer, gyroscope, accelerometer in three-axis, blood pressure, and foot pressure.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 shows a block diagram of an exemplary computing device, according to some embodiments of the invention;

FIG. 2 shows a block diagram of a system for determination of an activity status of a user with gait abnormality, according to some embodiments of the invention;

FIG. 3 schematically illustrates several examples of users with walking aids according to some embodiments of the invention;

FIG. 4 shows a flowchart of a method of determining status activity status of a user with gait abnormality, according to some embodiments of the invention; and

FIG. 5 shows a flowchart of a method of determining a mis-step of a user with gait abnormality, according to some embodiments of the invention.

It will be appreciated that, for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components, modules, units and/or circuits have not been described in detail so as not to obscure the invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing”, “computing”, “calculating”, “determining”, “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term set when used herein may include one or more items. Unless explicitly stated, the method embodiments described herein are not constrained to a particular order or sequence. Additionally, some of the described method embodiments or elements thereof can occur or be performed simultaneously, at the same point in time, or concurrently.

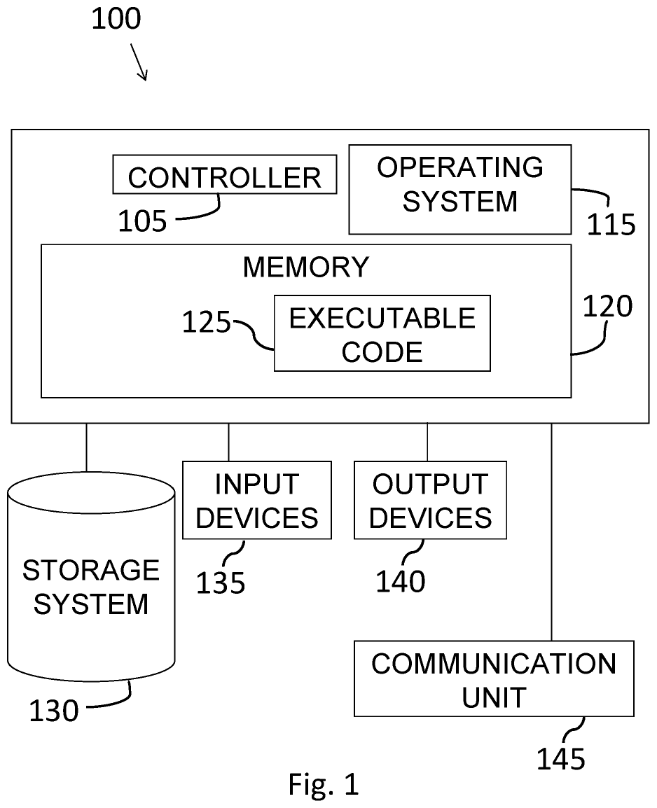

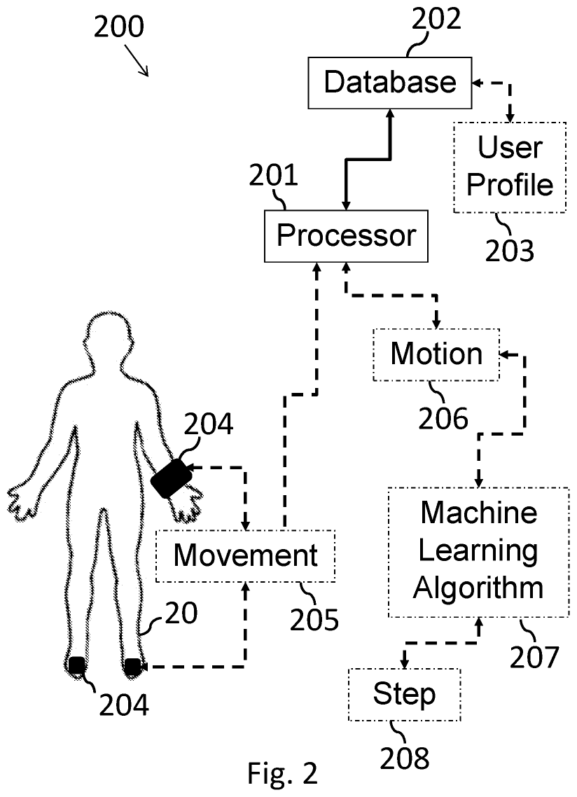

Reference is made to FIG. 1, which is a schematic block diagram of an example computing device 100, according to some embodiments of the invention. Computing device 100 may include a controller or processor 105 (e.g., a central processing unit processor (CPU), a programmable controller or any suitable computing or computational device), memory 120, storage 130, input devices 135 (e.g. a keyboard or touchscreen), and output devices 140 (e.g., a display), a communication unit 145 (e.g., a cellular transmitter or modem, a Wi-Fi communication unit, or the like) for communicating with remote devices via a communication network, such as, for example, the Internet. The computing device 100 may operate by executing an operating system 115 and/or executable code 125. Controller 105 may be configured to execute program code to perform operations described herein. The system described herein may include one or more computing device 100, for example, to act as the various devices or the components shown in FIG. 2. For example, system 200 may be, or may include computing device 100 or components thereof.

Operating system 115 may be or may include any code segment (e.g., one similar to executable code 125 described herein) designed and/or configured to perform tasks involving coordinating, scheduling, arbitrating, supervising, controlling or otherwise managing operation of computing device 100, for example, scheduling execution of software programs or enabling software programs or other modules or units to communicate.

Memory 120 may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 120 may be or may include a plurality of, possibly different memory units. Memory 120 may be a computer or processor non-transitory readable medium, or a computer non-transitory storage medium, e.g., a RAM.

Executable code 125 may be any executable code, e.g., an application, a program, a process, task or script. Executable code 125 may be executed by controller 105 possibly under control of operating system 115. For example, executable code 125 may be a software application that performs methods as further described herein. Although, for the sake of clarity, a single item of executable code 125 is shown in FIG. 1, a system according to some embodiments of the invention may include a plurality of executable code segments similar to executable code 125 that may be stored into memory 120 and cause controller 105 to carry out methods described herein.

Storage 130 may be or may include, for example, a hard disk drive, a universal serial bus (USB) device or other suitable removable and/or fixed storage unit. In some embodiments, some of the components shown in FIG. 1 may be omitted. For example, memory 120 may be a non-volatile memory having the storage capacity of storage 130. Accordingly, although shown as a separate component, storage 130 may be embedded or included in memory 120.

Input devices 135 may be or may include a keyboard, a touch screen or pad, one or more sensors or any other or additional suitable input device. Any suitable number of input devices 135 may be operatively connected to computing device 100. Output devices 140 may include one or more displays or monitors and/or any other suitable output devices. Any suitable number of output devices 140 may be operatively connected to computing device 100. Any applicable input/output (I/O) devices may be connected to computing device 100 as shown by blocks 135 and 140. For example, a wired or wireless network interface card (NIC), a universal serial bus (USB) device or external hard drive may be included in input devices 135 and/or output devices 140.

Some embodiments of the invention may include an article such as a computer or processor non-transitory readable medium, or a computer or processor non-transitory storage medium, such as for example a memory, a disk drive, or a USB flash memory, encoding, including or storing instructions, e.g., computer-executable instructions, which, when executed by a processor or controller, carry out methods disclosed herein. For example, an article may include a storage medium such as memory 120, computer-executable instructions such as executable code 125 and a controller such as controller 105. Such a non-transitory computer readable medium may be, for example, a memory, a disk drive, or a USB flash memory, encoding, including or storing instructions, e.g., computer-executable instructions, which, when executed by a processor or controller, carry out methods disclosed herein. The storage medium may include, but is not limited to, any type of disk including, semiconductor devices such as read-only memories (ROMs) and/or random-access memories (RAMs), flash memories, electrically erasable programmable read-only memories (EEPROMs) or any type of media suitable for storing electronic instructions, including programmable storage devices. For example, in some embodiments, memory 120 is a non-transitory machine-readable medium.

A system according to some embodiments of the invention may include components such as, but not limited to, a plurality of central processing units (CPU) or any other suitable multi-purpose or specific processors or controllers (e.g., controllers similar to controller 105), a plurality of input units, a plurality of output units, a plurality of memory units, and a plurality of storage units. A system may additionally include other suitable hardware components and/or software components. In some embodiments, a system may include or may be, for example, a personal computer, a desktop computer, a laptop computer, a workstation, a server computer, a network device, or any other suitable computing device. For example, a system as described herein may include one or more facility computing device 100 and one or more remote server computers in active communication with one or more facility computing device 100 such as computing device 100, and in active communication with one or more portable or mobile devices such as smartphones, tablets and the like.

According to some embodiments, systems and methods are provided for detection of daily activities (e.g., such as walking, sleeping, sitting down, standing up etc.) and/or counting of steps performed by elderly individuals or neurology patients (e.g., with walking aids) using machine learning algorithms. This detection may be performed based on signals from generic computing devices such as sensors of a smart-watch (e.g., acceleration and/or heart rate sensors).

Proper detection of such activities may also determine missteps. A misstep is defined as a poorly judged step which results in a non-fall event, where such missteps are typically predictors of falls by elderly users or neurology patients. Fall events may also include slipping, tripping and loss of balance.

Reference is now made to FIG. 2, which shows a block diagram of a system 200 for determination of an activity status of a user with gait abnormality, according to some embodiments. In FIG. 2, hardware elements are indicated with a solid line and the direction of arrows may indicate the direction of information flow.

The system 200 may include a processor 201 (e.g., such as controller 105 shown in FIG. 1) configured to execute a program (e.g., such as executable code 125 shown in FIG. 1) to determine an activity status of a user with gait abnormality. According to some embodiments, the processor 201 may be embedded in a mobile device such as a smartphone, tablet, etc. and/or embedded in a wearable device such as a smart-watch.

In some embodiments, the system 200 may include a database 202 (e.g., such as storage system 130 shown in FIG. 1), in active communication with the processor 201, where the database 202 may be configured to store one or more user profiles 203 for one or more users 20. For instance, the user profile 203 may include information with at least one of: medical history, medication used by the user, type of walking aid, social status, age, height, and location history.

In some embodiments, the system 200 may also include at least one wearable sensor 204, connected to the processor 201, and configured to detect a movement 205 of the user 20. For example, the at least one wearable sensor 204 may be embedded in a smart-watch, and/or a wristband and/or embedded as an insole sensor and/or in a chest patch. Signals from the at least one wearable sensor 204 may be received by the processor 201 for further analysis. In some embodiments, signals from the at least one wearable sensor 204 may be sampled at frequencies between 1 Hz to 100 Hz.

In some embodiments, the at least one wearable sensor 204 (e.g., worn on the wrist, or embedded into a shoe's sole) may allow measurements of user's posture and/or detect fall events. In some embodiments, the at least one wearable sensor 204 may perform processing (e.g., the heavy computations in the algorithm), for instance instead of processor 201, such that dependency on cloud-based computations may be reduced and thereby reduce data transfer needs to and from the cloud. For example, the processor 201 may train machine learning algorithms and download the resulted trained algorithm onto the at least one wearable sensor 204 for determination of mis-step and/or fall events.

In some embodiments, the at least one wearable sensor 204 may include at least one sensor of: heart rate, heart rate variability, galvanic skin response, electrocardiogram, SpO2, barometric pressure, magnetometer, gyroscope, accelerometer in one or more axes, blood pressure, and pressure sensors (e.g., in insole sensors). In some embodiments, measurement from the at least one wearable sensor 204 may include measurements of at least acceleration data (e.g., in three dimensional axes), for instance obtained by an accelerometer.

According to some embodiments, the processor 201 may be configured to identify a type of motion 206 carried out by at least one user 20. The type of motion 206 may be identified based on user profiles 203 received from the database 202 and/or base on data from the at least one wearable sensor 204 (e.g., data of determined movements 205).

According to some embodiments, the processor 201 may receive user metadata (e.g., obtained from a user upon first signup to the system), to be combined with measurements from the at least one wearable sensor 204 for identification of the type of motion 206. In some embodiments, the processor 201 may receive and/or pre-process time series data with temporal abstraction for varying sampling rates and combining user's metadata for a user-aware reference.

In some embodiments, the received user metadata may include type of walking aid device used (e.g., walking cane, stick, tripod, quadri-pod, walking frame, walking frame with wheels, cart, rollator, etc.). The received user metadata may also include details for the user profile 203 such as: weight, height, age, medications used by the user, history of illness etc.

In some embodiments, the processor 201 may receive tags of desired activity (e.g., walking) and/or other predefined activities, (e.g., obtained from manual tagging and/or sensor-based tagging), to be combined with measurements from the at least one wearable sensor 204 for identification of the type of motion 206. The manual tagging may be based on a dedicated mobile app that allows tagging of at least one of: gait sequences, non-gait sequences, single steps, mis-steps, falls, etc., and which also allows for annotation of events during those tagged sequences. Sensor based tagging may be based on the usage of one or more sensor measurements (e.g., from the at least one wearable sensor 204) to create tagged data and/or desired results for another sensor data. For example, using insoles pressure data to create labels for hand-band steps identification or using video recordings of gait sessions.

In some embodiments, the processor 201 may be configured to determine at least one step 208 carried out by the at least one user 20 using a machine learning algorithm 207 trained to determine steps 208 based on the identified motion 206. In some embodiments, the machine learning algorithm 207 may be trained to determine steps by classifying activity statuses based on a dataset of motion data for users with gait abnormality.

In some embodiments, the machine learning algorithm 207 may be configured to train a neural network to classify a given sample to having a predefined desired activity status (e.g., walking). The neural network may include at least one of Gated Recurrent Units (GRUs), Long-Short-Term-Memory (LSTM) neural networks, and/or Convolutional Neural Networks (CNNs), and/or Deep Auto Encoders (DAEs), and/or attention based neural networks, and/or transformer based neural networks, and/or gradient boosted decision tree algorithms (e.g., to improve accuracy of the outcome). In some embodiments, the machine learning algorithm 207 may generate a prediction of gait (e.g., as the baseline for normal activity status) for a specific user, based on previously received sensor data.

In some embodiments, the processor 201 may receive location data of the at least one wearable sensor 204 from location-based services (e.g., GPS) so as to determine if actual location change of the user 20 has occurred.

According to some embodiments, determination of steps 208 may include identification of user walking representation and/or clustering of similar walking patterns or gaits, in addition to characterization of each of the walking patterns according to a walking aid used (or lack of use). Training data, for instance for training the machine learning algorithm 207, may be collected from various users with and without walking aids (e.g., a walking stick, a walker, a quadri-pod, a cart, etc.). In some embodiments, the training may include manual tagging and/or sensors-based tagging.

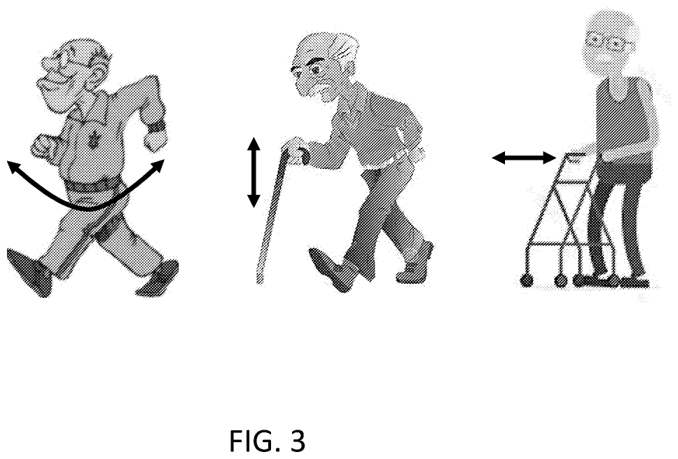

Reference is now made to FIG. 3, which schematically illustrates several examples of users with walking aids, according to some embodiments. It should be noted that the gait of the user with the walking aid, for instance due to gait abnormality, may be affected by the type of the walking aid (e.g., cane), with additional movement in the direction of the arrows shown in FIG. 3.

Reference is now made back to FIG. 2. According to some embodiments, the personal health user profile 203 of the elderly user or neurology patient 20 may be used in combination with sensor data regarding personal movements 205 and/or physiology signals (e.g., heart rate) to assess the periodic and/or accumulated user's steps. For example, the assessment may be carried out on a daily basis.

According to some embodiments, at least one of the following clustering methods may be used for modeling the user walking representation and/or clustering of similar walking patterns or gaits: classification, regression and/or point-wise segmentation. For classification, a combination of GRUs and CNN's may be used in order to create a classification for steps (e.g., taken by an elderly user) appearance in time slices of 1-10 seconds. This classification may accordingly be used to aggregate and/or separate the time sequences in which walking is observed from those with other activity (e.g., sleeping), or no activity at all. In some embodiments, similarity learning may be used for abstraction and/or representation of gait abnormality patterns.

For regression, a fully convolutional network may be used for the end-to-end task of counting steps (e.g., taken by an elderly user) observed within varying-length sequences of signals from the sensors 204. For segmentation, sensor-based tagging may be used in order to create a point-wise segmentation network for each measured point in time so as to segment its state affiliation to walking activity (or lack thereof) derived by the context of that measure.

In some embodiments, the at least one wearable sensor 204 may continuously collect data to be analyzed in order to determine the personal motion pattern of the user 20. For example, the personal amplitude of accelerometer signals may be analyzed while the user 20 is walking and the corresponding response of the heart rate to the walk may be detected, also taking in consideration whether the user 20 is using a cane or other walking aid. The processor 201 may apply the machine learning algorithm 207, for instance trained with the collected data, in order to match personal walking patterns or gaits with different individuals' personal history (e.g., from the user profile 203) and cluster these patterns to subgroups until an initial baseline may be determined for the user. For example, once the baseline for typical behavior (e.g., walking patterns) is determined, any movement exceeding this baseline may be monitored and/or issue an alert. In some embodiments, a report is issued (e.g., instead of an alert), the report including predicted and/or detected state such as misstep and/or fall. In some embodiments, the processor 201 may analyze the predicted state to determine the appropriate recipient (e.g., the user and/or a caregiver) of the issued report or alert. In some embodiments, upon issuing such alert, the recipient (e.g., the user and/or a caregiver) may automatically receive a call (e.g., via a predefined cellular service).

In some embodiments, short-term historical data (e.g., stored on the database 202) may be used to calculate a statistical baseline measures (e.g., specific percentile, fast furrier transform, range, etc.) per each user 20. In some embodiments, the measured baseline may be normalized for a predefined group of users (e.g., for users over 90 years old using a walker).

In some embodiments, the machine learning algorithm 207 may be trained on data from at least one of: anomaly detection algorithm (e.g., to extract potential anomalies from the wearable sensors' signal), received handcrafted features (e.g., from the wearable sensor) and gait classifier. In some embodiments, the anomalies detector may use low threshold to filter potential mis-steps, where the low threshold may be used within the anomaly detector network as the goal of this step is to filter out certain normal behaviors. In some embodiments, anomaly detection may be carried out using a multi sensor encoder-decoder network.

In some embodiments, the normalized signal may be used as training data and tags may be used as labels (e.g., with each training sample including 1-10 seconds of data). Each data sample may include a mixture of time series data along with the corresponding metadata and/or at least one label (e.g., gait/non-gait).

In some embodiments, the minimal information required by the processor 201 to properly determine the gait may be acceleration data (e.g., in three dimensional axes), for example with a specific model may not be trained using additional information (e.g., heart rate, gyro, metadata, etc.) and may be used to classify the status of the performed activity. In some embodiments, the processor 201 may segment or cluster users 20 using acceleration data and use a separate model per user-segment to replace the need of metadata and/or fit a more personalized model for gait detection.

The determined baseline may be continuously updated and/or improved with newly collected data for that user 20, for instance according to the personal walking pattern or gaits and/or physiological response to the walk. In some embodiments, by analyzing a large number of walking patterns from different users (e.g., for known walking movement of elderly users), a database of walking patterns or gaits and corresponding physiological responses may be created (e.g., stored on the database 202).

In some embodiments, a normal walking pattern or gait of the user 20 may be determined by calibrating the at least one sensor 204 in monitored conditions, for instance calibrating insole sensors to correspond with a dedicated mattress with a plurality of pressure sensors to determine the normal gait and make sure that the insole sensors provide similar results as obtained in lab conditions. Deviation from the calibrated walking pattern (or gait) may be tested by provoking an intentional misstep on the dedicated mattress. In case of identifying a movement that exceeds the calibrated baseline gait, an alert may be issued to the user and/or to a caregiver to assess preventive/rehab treatment and mitigate the observed growing risk.

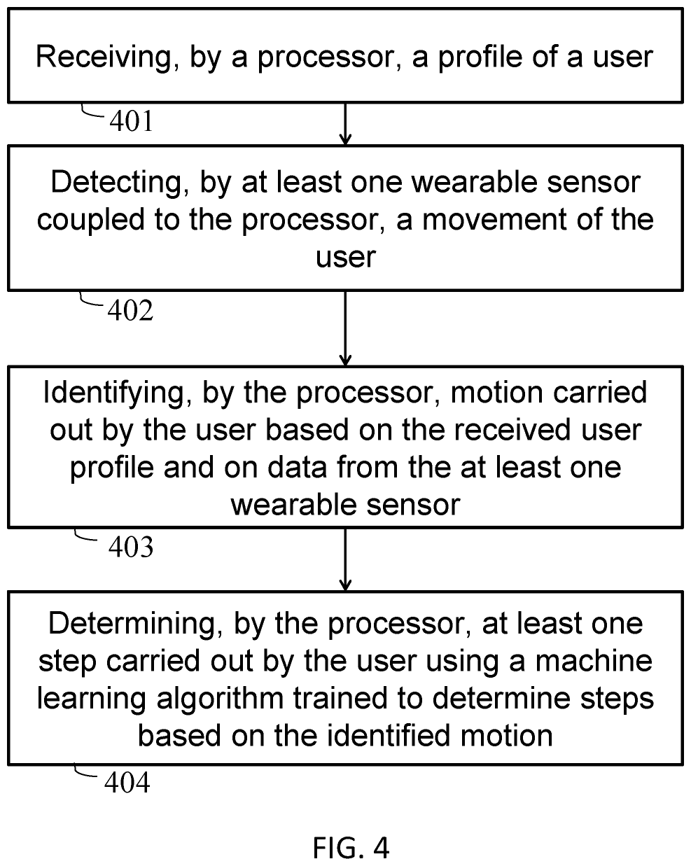

Reference is now made to FIG. 4, which shows a flowchart of a method of determining activity status of a user with gait abnormality, according to some embodiments. In Step 401, a profile of a user may be received (e.g., by the processor).

In Step 402, a movement of the user may be detected (e.g., by at least one wearable sensor coupled to the processor). In Step 403, motion carried out by the user may be identified (e.g., by the processor) based on the received user profile and on data from the at least one wearable sensor. In Step 404, at least one step carried out by the user may be determined (e.g., by the processor) using a machine teaming algorithm trained to determine steps based on the identified motion.

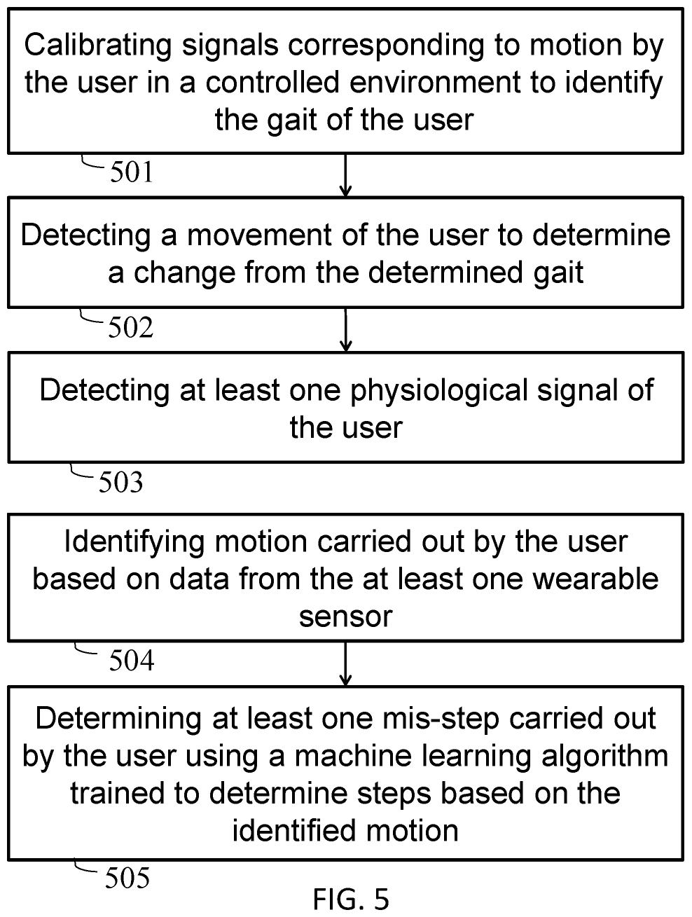

Reference is now made to FIG. 5, which shows a flowchart of a method of determining a mis-step of a user with gait abnormality, according to some embodiments. In Step 501, signals corresponding to motion by the user may be calibrated in a controlled environment to identify the gait of the user (e.g., by the processor).

In Step 502, a movement of the user may be detected (e.g., by at least one wearable sensor coupled to the processor) to determine a change from the determined gait. In Step 503, at least one physiological signal of the user may be detected (e.g., by the at least one wearable sensor). In Step 504, motion carried out by the user may be identified (e.g., by the processor) based on data from the at least one wearable sensor. In Step 505, at least one mis-step carried out by the user may be determined (e.g., by the processor) using a machine learning algorithm trained to determine steps based on the identified motion.

According to some embodiments, determined mis-step events may be used for prediction of fall events and/or additional physiological patterns. In some embodiments, escalation of user determined mis-step events (e.g., tagged by the user) may trigger prediction of a fall event, for instance compared to the user's past mis-step events over time.

According to some embodiments, detection of multiple mis-step events may be determined as an increased fall risk. According to some embodiments, detection of multiple fall events may be determined as an increased fall risk.

According to some embodiments, sleep duration and/or sleep quality of the user may be monitored in order to determine an increase in fall risk. The sleep monitoring may include monitoring sleep patterns of the user and/or analyzing historical sleep pattern data. For example, the sleep of the user may be monitored by monitoring movement of the user (e.g., via the wearable sensor) as well as monitoring of wakeups (e.g., registered by the user). According to some embodiments, number of wakeups of the user during a single night may be monitored in order to determine an increase in fall risk.

According to some embodiments, abnormal physiological and/or behavioral abnormalities of the user may be monitored (e.g., indicated by the user or by a caregiver) in order to determine an increase in fall risk. In some embodiments, detected movement pattern and/or physiological pattern changes may be correlated with medication changes (e.g., indicated by the user or by a caregiver) in order to determine an increase in fall risk.

While certain features of the invention have been illustrated and described herein, many modifications, substitutions, changes, and equivalents may occur to those skilled in the art. It is, therefore, to be understood that the appended claims are intended to cover all such modifications and changes as fall within the invention.

Various embodiments have been presented. Each of these embodiments may, of course, include features from other embodiments presented, and embodiments not specifically described may include various features described herein.

1. A method of determining activity status of a user with gait abnormality, the method comprising:
receiving, by a processor, a profile of the user;
detecting, by at least one wearable sensor coupled to the processor, a movement of the user;
identifying, by the processor, motion carried out by the user based on the received user profile and on data from the at least one wearable sensor;
training a machine learning algorithm to determine steps, wherein the training comprises applying a regressor to determine at least one step based on varying-length sequences of signals from the at least one wearable sensor;
performing abstraction of gait abnormality patterns using similarity learning; and
determining, by the processor, at least one step carried out by the user using the machine learning algorithm based on the identified motion.

2. The method of claim 1, wherein the at least one wearable sensor detects signals with information indicative of physiological status selected from the group consisting of: heart rate, heart rate variability, blood pressure, foot pressure, magnetometer, gyroscope, and accelerometer in three-axis.

3. The method of claim 1, wherein the user profile comprises information selected from the group consisting of: medical history, medication used by the user, type of walking aid, social status, age, height, and location history.

4. The method of claim 1, further comprising training the machine learning algorithm to determine steps, wherein the training comprises classifying activity statuses based on a dataset of motion data for users with gait abnormality.

5. (canceled)

6. The method of claim 1, further comprising training the machine learning algorithm to determine steps, wherein the training comprises creating a point-wise segmentation network for each measured point in time to determine the activity status based on the context of the measurement by the at least one wearable sensor.

7. (canceled)

8. (canceled)

9. The method of claim 1, further comprising clustering similar walking patterns sampled by other users with gait abnormality.

10.-15. (canceled)

16. A method of determining a mis-step of a user with gait abnormality, the method comprising:
calibrating, by a processor, signals corresponding to motion by the user in a controlled environment to identify the gait of the user;
detecting, by at least one wearable sensor coupled to the processor, a movement of the user to determine a change from the determined gait;
detecting, by the at least one wearable sensor, at least one physiological signal of the user;
identifying, by the processor, motion carried out by the user based on data from the at least one wearable sensor;
training a machine learning algorithm to determine steps, wherein the training comprises applying a regressor to determine at least one step based on varying-length sequences of signals from the at least one wearable sensor;
performing abstraction of gait abnormality patterns using similarity learning; and
determining, by the processor, at least one mis-step carried out by the user using the machine learning algorithm based on the identified motion.

17. The method of claim 16, further comprising training the machine learning algorithm to determine steps with a dataset of motion data for users with known attributes.

18. The method of claim 16, further comprising predicting a fall event based on at least one of: an escalation of determined mis-step events, and detection of multiple mis-step events.

19. (canceled)

20. The method of claim 16, further comprising predicting a fall risk based on at least one of: monitoring sleep of the user, and monitoring behavioral abnormalities of the user.

21. The method of claim 7, further comprising predicting a fall risk based on correlation between detected movement changes and medication changes of the user.

22. (canceled)

23. The method of claim 16, further comprising detecting, by the at least one wearable sensor, at least one of: mis-step by the user, and fall by the user.

24.-26. (canceled)

27. A system for determination of a mis-step of a user with gait abnormality, the system comprising:
a database, comprising a calibrated set of signals corresponding to gait of the user;
at least one wearable sensor, configured to:
detect a movement of the user; and
detect at least one physiological signal of the user; and
a processor, coupled to the database and to the at least one wearable sensor, and configured to:
determine a change from the calibrated gait;
identify a motion carried out by the user based on the received user profile and on signal from the at least one wearable sensor;
perform abstraction of gait abnormality patterns using similarity learning; and
determine at least one mis-step carried out by the user using a machine learning algorithm trained to determine steps based on the identified motion,
wherein the machine learning algorithm is trained to determine steps, and wherein the training comprises applying a regressor to determine at least one step based on varying-length sequences of signals from the at least one wearable sensor.

28. The system of claim 27, wherein the machine learning algorithm is trained to determine steps with a dataset of motion data for users with known attributes.

29. The system of claim 28, wherein the known attributes comprise information selected from the group consisting of: medical history, medication used by the user, type of walking aid, social status, age, height, and location history.

30. The system of claim 27, wherein the processor is configured to predict a fall event based on at least one of: an escalation of determined mis-step events, and detection of multiple mis-step events.

31. (canceled)

32. The system of claim 27, wherein the processor is configured to predict a fall risk based on at least one of: monitoring sleep of the user, and monitoring behavioral abnormalities of the user.

33. The system of claim 27, wherein the processor is configured to predict a fall risk based on correlation between detected movement changes and medication changes of the user.

34. (canceled)

35. The system of claim 27, wherein the at least one wearable sensor is configured to detect at least one of: fall by the user, and mis-step by the user.

36. (canceled)

37. The system of claim 27, wherein the at least one wearable sensor comprises at least one of a smartwatch motion-sensor and an insole sensor.

38. (canceled)
